(19) United States
(12) Patent Application Publication
(10) Pub. No.: US 2021/0038149 A1
(43) Pub. Date: Feb. 11, 2021

(54) Blood alcohol monitoring system calibrated for the individual guaranteeing the BAC Sweet Spot is maintained

(71) Applicant: Tyler Michael Graves, Framingham, MA (US)

(72) Inventor: Tyler Michael Graves, Framingham, MA (US)

(21) Appl. No.: 16/537,511

(22) Filed: Aug. 9, 2019

(51) Int. Cl. A61B 5/00; G08B 7/06; A61B 5/145; B60R 25/04

(52) U.S. Cl. CPC A61B 5/4845; G08B 7/06; A61B 5/7275; A61B 5/743; H04L 67/1097; A61B 5/14546; A61B 5/7455; B60R 25/04; A61B 5/0022

(57) ABSTRACT

The disclosed system, which includes a wearable BAC (Blood Alcohol Content) sampling device and proprietary monitoring software, provides the wearer with the confidence to drink alcoholic beverages freely, up to the point where the software determines the wearer's BAC will be approaching the BAC Sweet Spot. The BAC Sweet Spot is defined as a range of BAC levels at which a person should be experiencing the height of the alcoholic experience, but at the same time not overindulging. The precise personalized calculation of the BAC Sweet Spot and maintaining the users BAC within the BAC Sweet Spot throughout the drinking session are the goals of this invention.

### FIELD OF INVENTION

The technology relates to the general field of biomedical devices, and has certain specific applications to personal safety and mental health.

### BACKGROUND

Alcohol consumption management programs and measurement devices similar to U.S. Pat. No. 4,057,724 are plentiful in the marketplace, and many will reduce over-consumption and save lives. The development of an inexpensive, portable breathalyzer enables people to nearly instantly and conveniently verify their BAC (Blood Alcohol Content) before operating a motor vehicle or other machinery. A major drawback to most of these devices is the reliance on (potentially) intoxicated individuals to perform routine samplings of their BAC. Passive monitoring mechanisms, such as a transdermal sensor similar to U.S. Ser. No. 10/441,940, can be combined with a monitoring and alerting mechanism to provide a more robust solution.

Monitoring and alerting based on a specific BAC maximum is the most basic use of these devices. Enhancing these basic BAC monitoring devices with enhanced data analysis and prediction algorithms can greatly increase their adoption rate and benefits to the end user.

### The Invention

The system disclosed here is a hardware and software solution. The hardware leverages familiar devices such as transdermal alcohol metering and display drivers with touch screens. The design of the management, monitoring and sample analysis software routines and their application are novel. Enabling a consumer of alcoholic beverages the safety and security of maintaining an ideal BAC (Blood Alcohol Content) level is unique, compared to typical systems that simply report back when a particular maximum BAC is detected. With those basic systems, the BAC level is not customized for the user's optimum alcohol consumption experience and more likely a simple maximum value legislated by an automobile operation maximum BAC (typically 0.08).

This software invention determines when the wearer's BAC will be approaching the BAC Sweet Spot. The BAC Sweet Spot is defined as a range of BAC levels at which a person should be experiencing the height of the alcoholic experience, but at the same time not overindulging. The precise personalized calculation of the BAC Sweet Spot and maintaining the users BAC within the BAC Sweet Spot throughout the drinking session are the primary goals of this invention.

The definition of the user's BAC Sweet Spot is continuously refined throughout the use of the device, and progressively gets more personalized over time. A typical value for the BAC Sweet Spot is initially set, but that is intended only as a starting point. As the user drinks alcoholic beverages, he or she will periodically press the thumbs up or thumbs down buttons to record their current intoxication enjoyment level. As the drinking session commences, quite frequently the sensation of feeling good will peak, then tail off as the user either reduces their consumption rate or exceeds the upper boundary of the BAC Sweet Spot and into overindulgence. A typical drinking session could comprise of a series of thumbs up user input, followed by a lull in data or possibly thumbs down user input (in the event of overindulgence). If the alcohol consumption session is long in duration, several oscillations of feeling good, feeling bad, feeling good may be recorded. With these data, the device can now more accurately determine the BAC Sweet Spot and provide feedback to the user to ultimately avoid periods of feeling bad or overindulgence.

A state machine enables the measuring system to periodically sample the wearer's BAC and submit the latest readings to a trend analysis engine. The trend analysis engine then evaluates the current BAC along with historical data to make a determination if the user is either below, above or within the BAC Sweet Spot. An alerting and display driver module will then be engaged to provide updated status to the user, depending on the user preference settings.

Displaying the BAC trend overlayed with the BAC Swett Spot range allows to user to visually chart the BAC changes real time.

In the event the software determines a user is below the BAC Sweet Spot, the software will determine the suggested number of beverages the wearer can additionally consume in order to reach the BAC Sweet Spot. The number of beverages is dependent on the type of drink being consumed, and that can be adjusted through the user interface by the user to reflect the correct drink type.

In the event the software determines a user is above the BAC Sweet Spot, the wearer is alerted that they must stop drinking for a prescribed amount of time in order to lower their BAC and return to the BAC Sweet Spot range. The duration of time needed is highly dependent on the user's physical characteristics. Typical BAC monitoring systems do not include such personalization. This invention is unique in that it can leverage a historical database of BAC readings over time, and use that information to accurately predict the user's alcohol processing rate.

The software will determine through trend analysis calculations and historical data if the user will overshoot the BAC Sweet Spot range. The BAC Sweet Spot value and the rate at which the wearer approaches the BAC Sweet Spot is determined through specialized calculations taking into account variables such as standard human physiology, analysis of historical beverage consumption data and direct input about their perceived level of intoxication from the wearer.

A social media and friend module, combined with a cloud adapter, enables the system to communicate with external resources. These resources could be other BAC monitor systems or social media providers. With this level of communications, the user can share their current BAC with other BAC monitoring systems for the purposes of drinking games for example. Another useful feature of this ability would be remote monitoring for safety. If a user's BAC reaches a set level, someone could be notified of this condition and take action if needed.

Standard BAC level alerts are available to enable or disable as desired, which can include 0.08 for typical USA automobile operation legal limits. Custom BAC levels can also be programmed into the unit for alerting purposes, if for example a desire to maintain a BAC below 0.04 due to additional constraints on legal limits for commercial vehicle operation.

### BRIEF DESCRIPTION OF THE DRAWINGS

Various embodiments of the invention are disclosed in the following detailed description and accompanying drawings.

FIG. 1 illustrates a system diagram.

FIG. 2 illustrates the state machine of the BAC Sweet Spot software and user feedback mechanisms.

FIG. 3 illustrates a wearable device user interface and display.

### DETAILED DESCRIPTION OF EMBODIMENTS

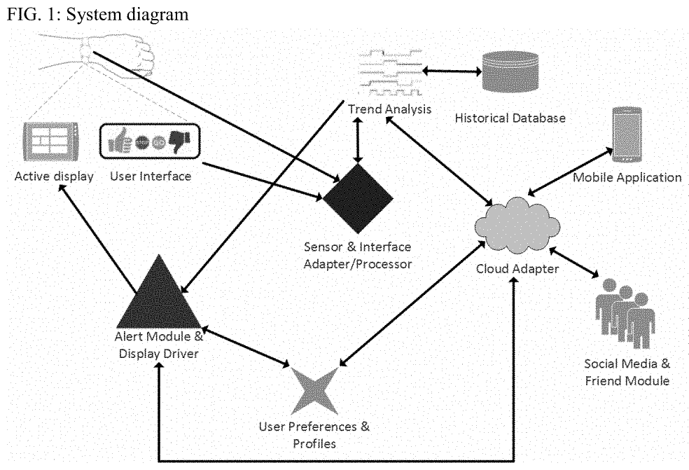

System component list details (see FIG. 1)

Trend Analysis Engine
1. Inputs: Current BAC sample, past BAC samples for current session, current BAC trend projection, trend analysis modifiers such as beverage type, Thumbs Up/Down intoxication comfort modifiers.
2. Outputs: Updated BAC trend projection

Historical Database
1. Retains previous BAC samples during previous monitoring periods
2. Thumbs up/down events and corresponding BAC sample with time/date details
3. Current and past BAC trending analysis results

Sensor & Interface Adapter/Processor
1. Monitors the user interface Thumbs Up/Down controls for user input and relays any input to the trend analysis engine
2. Drives the BAC sensor sampling process and relays the sensor data to the trend analysis engine for further processing

Cloud Adapter
1. Provides a consumable interface for internal modules to publish and receive data from external modules

Mobile Application
1. A custom application intended for use on mobile devices, enabling the user to control the wrist sensor through a mobile device and view more detailed information that what can be displayed on the wrist sensor's smaller active display
2. Provides an internet gateway for the wrist sensor's underlying cloud adapter module, enabling external communication capabilities

Social Media & Friend Module
1. Persists a list of Friends which are secure associations with other users' wrist sensors.
2. Friends have full control over what level of data is shared with other users, and associations can be temporary for a time-boxed drinking session or permanent until revoked.
3. Friend associations can be generated automatically with geolocation-type services, which would enable breweries or bars to setup zones where visitors are automatically grouped for the purposes of sharing each other's BAC levels.
4. When used in conjunction with the Mobile Application, a virtual map can be generated to present the BAC of friends in the area. This can enable the user to either avoid or gather with friends with high or low BAC levels at their own discretion.
5. Enables device-to-device communication between linked users (Friends), for the purposes of drinking games, monitoring another person's drinking habits, etc.

User Preferences & Profiles Manager
1. System preference settings, such as alert settings and contact information
2. For supporting multiple users, profile selection and management

Alert Module and Active Display Driver
1. A display driver for the wrist sensor's Active Display
2. This module is responsible for generating user alerts based on the combination of user preferences and the incoming data from the trend analysis engine
3. Alerts are generated based on input from the social media module or the mobile application

User interface and display
1. Input: Active touch interface for system configuration
2. Output: Active display for alerts and live BAC trending graph
3. Thumbs up and Thumbs down buttons for easy user input of current intoxication level
4. Stop and Go display for quick confirmation to halt or commence drinking

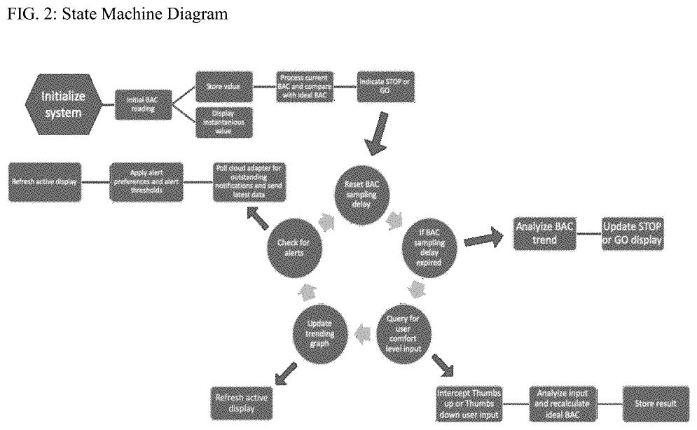

State machine diagram (see FIG. 2)

Typical state machine with user interaction with the system

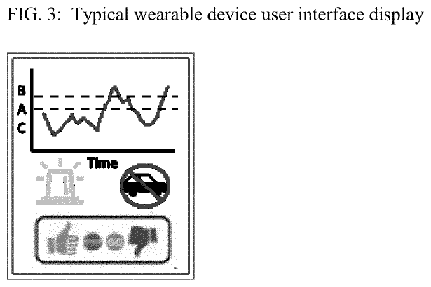

Typical wearable device user interface display details (see FIG. 3)

The wearable device could feature one or many of the user interface design characteristics such as
1. BAC graph over time, with indications where the BAC Sweet Spot is in relation to current BAC values.
2. Include projected BAC values on the BAC graph when possible to calculate a trend.
3. Active alerts
4. BAC over 0.08 notification, indicating unsafe to drive in most locations in the USA
5. Thumbs Up and Thumbs Down buttons for reporting current feelings of intoxication
6. Stop or Go sign indications for indicating if it is suggested to stop drinking or continue drinking for targeting the BAC Sweet Spot.

Typical Wearable Device Ideal Physical Design Characteristics (See FIG. 3)

The wearable device could feature one or many of the physical device design characteristics such as
1. Comfortable wrist strap, but also tight enough to maintain necessary skin contact for BAC sampling
2. Large enough Active Display screen so touch interface is easy to use
3. Display is easily viewed from direct view, but at the same time difficult enough to conceal the data displayed from side angles for privacy.

What is claimed is:

1. A method for achieving and maintaining a user's BAC (Blood Alcohol Content), for purposes of maximizing enjoyment of drinking alcoholic beverages, comprising of:
The sampling of the user's current BAC;
Processing the BAC level which results in displaying the current level;
The current BAC level is then applied to the novel BAC prediction algorithms and trend analysis consisting of linear prediction, extrapolation and the like;
The resulting analysis is then augmented with hysteresis, referencing past trends and user specific BAC ramp-up and ramp-down (alcohol processing) rates;
An easy to understand concept called the BAC Sweet Spot, which is defined as the range of BAC levels which maximize the good feelings brought on by drinking alcoholic beverages without overindulgence;
Display of the analysis results to the user in an easy to interpret display utilizing a familiar Stop and Go traffic light format, indicating if the user can continue to consume more alcohol;
Display of the quantity and type of drink which can be consumed before the BAC Sweet Spot is reached;
Retention of historical data archiving for the purposes of: developing a long-term alcohol consumption record, further enhancing the prediction algorithm by providing historical data to the trend analysis, cloud storage for backup and online access, mobile device integration, vehicle ignition interlock integration, third-party app integration with Beer/Wine tracking and rating applications to provide more precise input into consumed ABV of drinks, reporting current BAC to external entities for safety and monitoring, friend sharing and social media/drinking games;
Continuous refinement of the personalized BAC Sweet Spot as the user drinks beverages through user inputting their current state of intoxication comfort using a thumbs up/down button interface.

2. A device worn on the wrist to measure the current BAC of the wearer, accept direct input from the user and report various statistics to the user, comprising of:
A sensor for sampling the BAC via transdermal measurement;
A sampling mechanism capable of sampling BAC changes within a timeframe that would allow the user time to react and adjust consumption according to device notification;
Visual and haptic feedback status reporting: current BAC, BAC trending graph, BAC Sweet Spot in relation to current BAC, haptic feedback when trending towards a BAC Sweet Spot overshoot, Go/Stop consumption indication for BAC Sweet Spot targeting;
Simple user interface controls including features that allow: hide/show display for privacy, thumbs Up/Down input for current intoxication comfort level and beverage type selection.
